(19) United States
(12) Patent Application Publication
(10) Pub. No.: US 2007/0055224 A1
(43) Pub. Date: Mar. 8, 2007

(54) Intralumenal microwave device

(76) Inventors: Fred T. Lee JR., Madison, WI (US); Christopher L. Brace, Middleton, WI (US); Paul F. Laeseke, Madison, WI (US); Daniel Warren van der Weide, Madison, WI (US); Deepak Gopal, Madison, WI (US); Patrick Pfau, Madison, WI (US); Lisa A. Sampson, Madison, WI (US)

Correspondence Address: Patula & Associates, P.C., 14th Floor, 116 S. Michigan Ave., Chicago, IL, 60603, US

(21) Appl. No.: 11/502,783

(22) Filed: Aug. 11, 2006

Related U.S. Application Data
(63) Continuation-in-part of application No. 10/834,802, filed on Apr. 29, 2004, now Pat. No. 7,101,369.
(63) Continuation-in-part of application No. 11/237,136, filed on Sep. 28, 2005.
(63) Continuation-in-part of application No. 11/237,430, filed on Sep. 28, 2005.
(63) Continuation-in-part of application No. 11/236,985, filed on Sep. 28, 2005.
(63) Continuation-in-part of application No. 11/440,331, filed on May 24, 2006.
(63) Continuation-in-part of application No. 11/452,637, filed on Jun. 14, 2006.
(60) Provisional application No. 60/707,797, filed on Aug. 12, 2005.
(60) Provisional application No. 60/710,276, filed on Aug. 22, 2005.
(60) Provisional application No. 60/710,815, filed on Aug. 24, 2005.

(51) Int. Cl. A61B 18/18; A61F 2/00; A61N 1/00

(52) U.S. Cl. 606/33; 607/101; 607/156

(57) ABSTRACT

A microwave device that can be used to effectively treat esophageal pathologies comprises a coaxial, triaxial or quadraxial microwave antenna housed in an esophageal dilator or balloon. The device can be introduced into the esophagus alongside or through an endoscope, and will deliver microwave energy to tissue. This energy heats the affected tissue, which subsequently undergoes necrosis thereby eliminating the potential of the tissue to undergo malignant transformation. The dilator or balloon is used to keep the antenna in the center of the lumen allowing for symmetrical heating of the esophagus. The depth of penetration of the coagulation effect can be varied depending on the amount of power that is applied, the location of the antenna relative to the tissue, and the duration of the power application.

### CLAIM OF PRIORITY

This application is a Continuation-In-Part of co-pending U.S. Non-Provisional Patent Applications entitled “Triaxial Antenna for Microwave Tissue Ablation” filed Apr. 29, 2004 and assigned U.S. application Ser. No. 10/834,802; “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; “Air-Core Microwave Ablation Antennas” filed Sep. 28, 2005 and assigned U.S. application Ser. No. 11/236,985; “Microwave Surgical Device” filed May 24, 2006 and assigned U.S. application Ser. No. 11/440,331; and “Microwave Tissue Resection Tool” filed Jun. 14, 2006 and assigned U.S. application Ser. No. 11/452,637; the entire disclosures of each and all of these applications are hereby herein incorporated by reference.

This application further claims priority, where applicable, to U.S. Provisional Patent Applications entitled “Segmented Catheter for Tissue Ablation” filed May 10, 2005 and assigned U.S. application Ser. No. 60/679,722; “Microwave Surgical Device” filed May 24, 2005 and assigned U.S. application Ser. No. 60/684,065; “Microwave Tissue Resection Tool” filed Jun. 14, 2005 and assigned U.S. application Ser. No. 60/690,370; “Cannula Cooling and Positioning Device” filed Jul. 25, 2005 and assigned U.S. application Ser. No. 60/702,393; “Intralumenal Microwave Device” filed Aug. 12, 2005 and assigned U.S. application Ser. No. 60/707,797; “Air-Core Microwave Ablation Antennas” filed Aug. 22, 2005 and assigned U.S. application Ser. No. 60/710,276; and “Microwave Device for Vascular Ablation” filed Aug. 24, 2005 and assigned U.S. application Ser. No. 60/710,815; the entire disclosures of each and all of these applications are hereby herein incorporated by reference.

### CROSS-REFERENCE TO RELATED APPLICATIONS

This application is related to co-pending U.S. Non-Provisional Patent Applications entitled “Triaxial Antenna for Microwave Tissue Ablation” filed Apr. 29, 2004 and assigned U.S. application Ser. No. 10/834,802; “Segmented Catheter for Tissue Ablation” filed Sep. 28, 2005 and assigned U.S. application Ser. No. 11/237,136; “Cannula Cooling and Positioning Device” filed Sep. 28, 2005 and assigned U.S. application Ser. No. 11/237,430; “Air-Core Microwave Ablation Antennas” filed Sep. 28, 2005 and assigned U.S. application Ser. No. 11/236,985; “Microwave Surgical Device” filed May 24, 2006 and assigned U.S. application Ser. No. 11/440,331; and “Microwave Tissue Resection Tool” filed Jun. 14, 2006 and assigned U.S. application Ser. No. 11/452,637; and to U.S. Provisional Patent Applications entitled “Segmented Catheter for Tissue Ablation” filed May 10, 2005 and assigned U.S. application Ser. No. 60/679,722; “Microwave Surgical Device” filed May 24, 2005 and assigned U.S. application Ser. No. 60/684,065; “Microwave Tissue Resection Tool” filed Jun. 14, 2005 and assigned U.S. application Ser. No. 60/690,370; “Cannula Cooling and Positioning Device” filed Jul. 25, 2005 and assigned U.S. application Ser. No. 60/702,393; “Intralumenal Microwave Device” filed Aug. 12, 2005 and assigned U.S. application Ser. No. 60/707,797; “Air-Core Microwave Ablation Antennas” filed Aug. 22, 2005 and assigned U.S. application Ser. No. 60/710,276; and “Microwave Device for Vascular Ablation” filed Aug. 24, 2005 and assigned U.S. application Ser. No. 60/710,815; the entire disclosures of each and all of these applications are hereby herein incorporated by reference.

### FIELD OF INVENTION

The present disclosure relates generally to medical devices, and in particular, to medical devices in the field of radio-frequency (RF) ablation and/or microwave ablation. Specifically, the present disclosure relates to an intralumenal microwave device, for use for example to treat esophageal pathologies, and a method for intraluminal tissue ablation.

### BACKGROUND

Barrett's Esophagus is a precancerous condition of the esophagus that can progress to a type of cancer called esophageal adenocarcinoma. Barrett's esophagus is estimated to affect about 700,000 adults in the United States, and is associated with the very common condition gastroesophageal reflux disease or GERD. The risk of developing adenocarcinoma is 30 to 125 times higher in people who have Barrett's esophagus than in people who do not. While many people with Barrett's are asymptomatic and most will never progress to cancer, esophageal adenocarcinoma is often deadly as the condition is usually diagnosed late and the current treatments are not effective. Therefore, a treatment for Barrett's is needed that can effectively reduce the number of people that progress to adenocarcinoma without exposing asymptomatic people to unnecessary procedural complications and associated morbidity. The present disclosure fulfills this need.

### SUMMARY

This present disclosure relates to a microwave device that can be used for intraluminal tissue ablation, for example to effectively treat esophageal pathology, including (but not limited to) Barrett's Esophagus and esophageal adenocarcinoma. The preferred embodiment comprises a coaxial, triaxial or quadraxial microwave antenna housed in an esophageal dilator or balloon (FIGS. 1 and 3). The proposed device can be introduced into the esophagus alongside or through an endoscope, and will deliver microwave energy to tissue. This energy heats the affected tissue, which subsequently undergoes necrosis thereby eliminating the potential of the tissue to undergo malignant transformation. The dilator or balloon is used to keep the antenna in the center of or approximate the center of the lumen allowing for generally symmetrical heating of the esophagus.

Other permutations of the preferred embodiment are possible. For example, the antenna need not be a triaxial antenna. Various microwave antennas could be used to heat the tissue, and other mechanisms of positioning the antenna in the center of the lumen are possible. It is also possible that heating elements could be incorporated into the dilator or balloon itself such that the heating occurs closer to the tissue. Similarly, the antenna(s) could be placed in close proximity to the tissue during the ablation (e.g. using a spiral shaped antenna) to treat the tissue.

This device is different than current devices that are used. For instance, this device will run in the microwave spectrum and receive power from a microwave generator rather than radiofrequency energy or lasers. The preferred frequencies would be 915 MHz and 2.45 GHz, but other frequencies could also be used. The depth of penetration of the coagulation effect can be varied depending on the amount of power that is applied, the location of the antenna relative to the tissue, and the duration of the power application (FIG. 2).

Accordingly, it is one of the objects of the present disclosure to provide a method and device for intralumenal tissue ablation.

It is another object of the present invention to provide a method and device to treat esophageal pathologies.

It is a further object of the present invention to provide a microwave device and method for intralumenal introduction and delivery of microwave energy to tissue.

Numerous other advantages and features of the disclosure will become readily apparent from the following detailed description, from the claims and from the accompanying drawings in which like numerals are employed to designate like parts throughout the same.

### BRIEF DESCRIPTION OF THE DRAWINGS

A fuller understanding of the foregoing may be had by reference to the accompanying drawings wherein:

FIG. 1 is a picture of an esophageal dilator, a dilator balloon, and a microwave ablation antenna used in the device of the preferred embodiment of the present disclosure.

FIG. 2 is a chart illustrating the performance of the device of the preferred embodiment of the present disclosure.

FIG. 3 is a schematic diagram of the device of the preferred embodiment of the present disclosure in use in an esophagus using the balloon (left), and using the dilator (right).

### DESCRIPTION OF DISCLOSED EMBODIMENT(S)

While the invention is susceptible of embodiment in many different forms, there is shown in the drawings and will be described herein in detail one or more embodiments of the present disclosure. It should be understood, however, that the present disclosure is to be considered an exemplification of the principles of the invention, and the embodiment(s) illustrated is/are not intended to limit the spirit and scope of the invention and/or the claims herein.

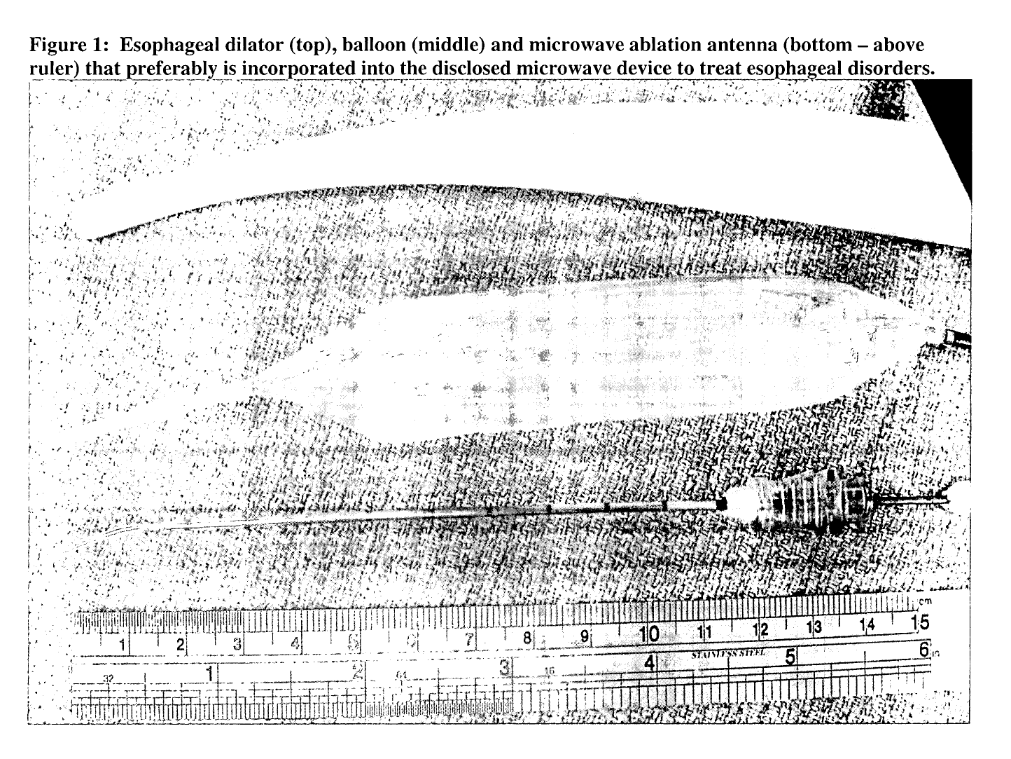

This present disclosure illustrates a microwave device that can be used to effectively treat esophageal pathology, including (but not limited to) Barrett's Esophagus and esophageal adenocarcinoma. The preferred embodiment comprises a coaxial, triaxial or quadraxial microwave antenna (as seen at the bottom of FIG. 1—above the ruler), which is housed in either an esophageal dilator (as seen at the top of FIG. 1) or a balloon (as seen in the middle of FIG. 1—between the dilator and the antenna) when in use. FIG. 1 shows the antenna separate from the dilator and the balloon.

This device is different than current devices that are used. For instance, this device will run in the microwave spectrum and receive power from a microwave generator rather than radiofrequency energy or lasers. The preferred frequencies would be 915 MHz and 2.45 GHz, but other frequencies could also be used.

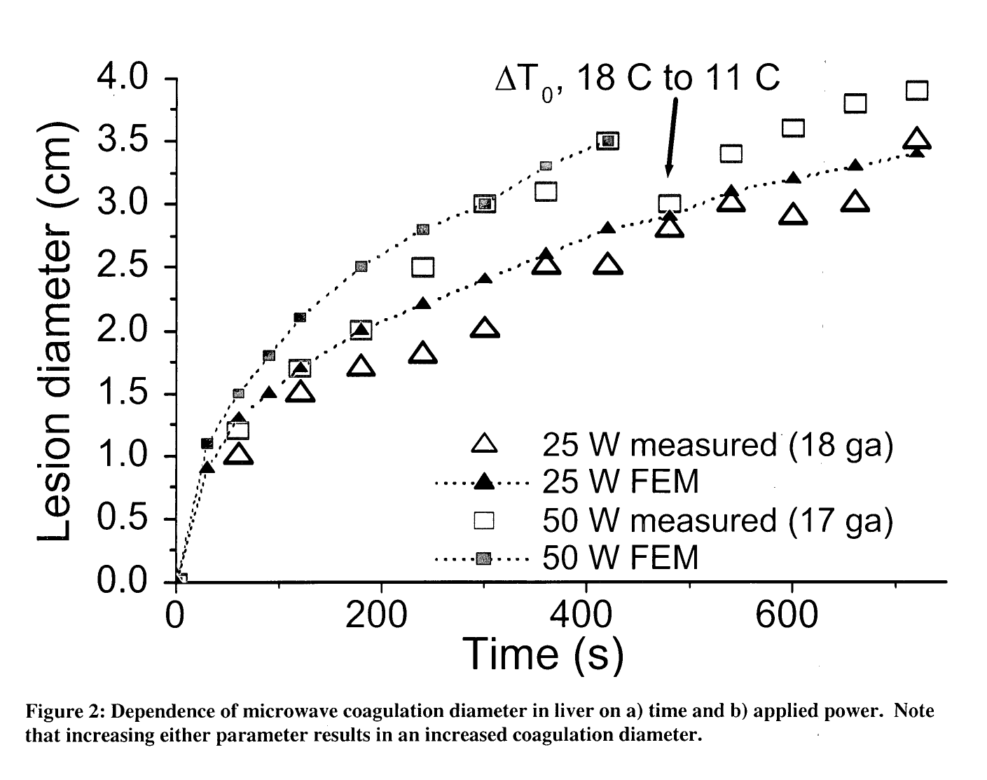

As illustrated in FIG. 2, the depth of penetration of the coagulation effect can be varied depending on the amount of power that is applied, the location of the antenna relative to the tissue, and the duration of the power application. The chart in FIG. 2 illustrates the dependence of microwave coagulation diameter or lesion diameter in liver on a) time and b) applied power. It is noted that increasing either parameter (time or power) results in an increased coagulation diameter.

The proposed device can be introduced into the esophagus alongside or through an endoscope, and will deliver microwave energy to tissue. This energy heats the affected tissue, which subsequently undergoes necrosis thereby eliminating the potential of the tissue to undergo malignant transformation.

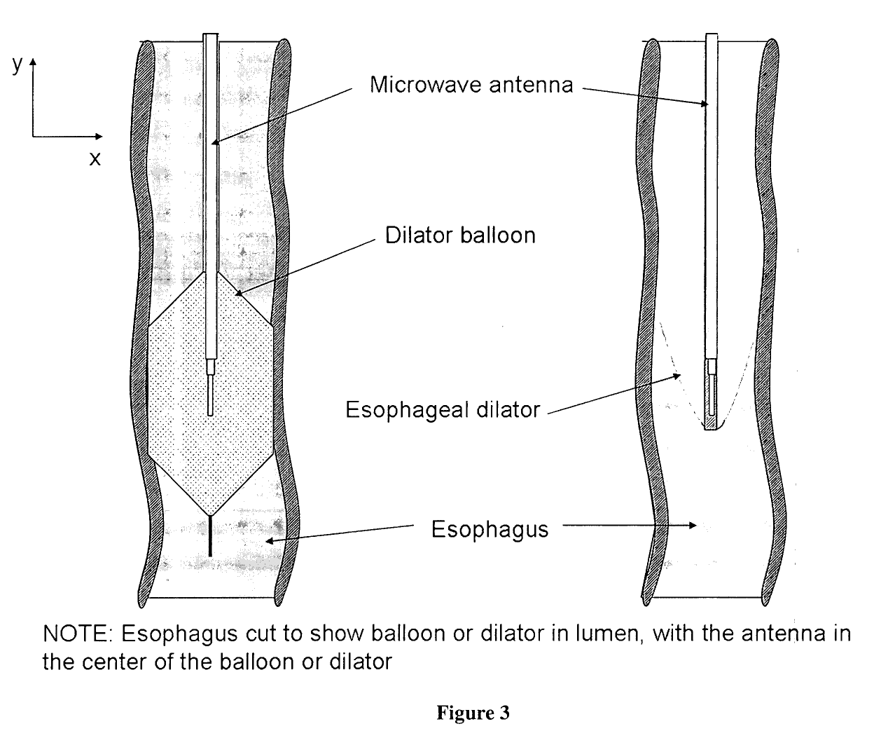

As can be seen in FIG. 3, the esophageal dilator (right side diagram) or the dilator balloon (left side diagram) is used to keep the antenna in the center of or proximate the center of the lumen allowing for generally symmetrical heating of the esophagus. Microwave energy can be fed to the antenna with a coaxial transmission line or dielectric or hollow-pipe waveguide. The applicator beneficially does not require conductive contact to the tissue under treatment.

It should be understood based upon the present disclosure that other permutations or modifications of the preferred embodiment are possible. For example, the antenna need not be a triaxial antenna. Various microwave antennas could be used to heat the tissue, and other mechanisms of positioning the antenna in the center of the lumen are possible and contemplated. It is also possible that heating elements could be incorporated directly into the dilator or balloon itself such that the heating occurs closer to the tissue. Similarly, the antenna(s) could be placed in close proximity to the tissue during the ablation (e.g. using a spiral shaped antenna) to treat the tissue. In general, any suitable power supply and microwave applicator combination for treatment of esophageal pathologies or other pathologies that can be introduced into a lumen through a breathing tube, a balloon dilator or any other like device is contemplated.

It is to be understood that the embodiment(s) herein described is/are merely illustrative of the principles of the present invention. Various modifications may be made by those skilled in the art without departing from the spirit or scope of the claims which follow.

What is claimed is:

1. A microwave applicator for treatment of esophageal pathologies, comprising:
a coaxial, triaxial, quadraxial, or similar resonant structure introduced to tissue under treatment through a breathing tube or balloon dilator;
a coaxial transmission line or dielectric or hollow-pipe waveguide feeding microwave power to the coaxial, triaxial, quadraxial, or similar resonant structure;
wherein the coaxial, triaxial, quadraxial, or similar resonant structure does not require conductive contact to the tissue under treatment.

2. An intralumenal microwave device for treatment of esophageal pathologies comprising:
a microwave power supply;
a microwave applicator; and
a breathing tube or balloon dilator through which the microwave applicator is introduced to tissue under treatment.

3. The device of claim 2, wherein the breathing tube or balloon dilator positions the microwave applicator proximate the center of the lumen allowing for generally symmetrical heating of the esophagus.

4. The device of claim 2, wherein the applicator does not require conductive contact to the tissue under treatment.

5. A method for intralumenal tissue ablation, comprising the steps of:
introducing a coaxial, triaxial, quadraxial, or similar resonant structure to tissue under treatment through a breathing tube or balloon dilator; and
supplying microwave power to the coaxial, triaxial, quadraxial, or similar resonant structure to ablate the tissue under treatment.

6. The method of claim 5, further comprising the step of varying a depth of penetration of coagulation effect on the tissue under treatment by varying at least one of the amount of power that is applied, the location of the structure relative to the tissue, and the duration of the power application.

7. The method of claim 5, further comprising the step of centering the coaxial, triaxial, quadraxial, or similar resonant structure in the breathing tube or balloon dilator.
